(19) United States
(12) Patent Application Publication
(10) Pub. No.: US 2022/0205966 A1
(43) Pub. Date: Jun. 30, 2022

(54) METHOD AND APPARATUS FOR MONITORING QUALITY AND DETERMINING CONTAMINATION OF AN AREA

(71) Applicant: CURRENTA GMBH & CO. OHG, Leverkusen (DE)

(72) Inventors: Marc André ALTHOFF, Köln (DE); Axel VON STUMBERG, Bergisch Gladbach (DE)

(21) Appl. No.: 17/601,455

(22) PCT Filed: Jul. 20, 2020
(86) PCT No.: PCT/EP2020/070408

§ 371 Date: Oct. 5, 2021

(30) Foreign Application Priority Data
Jul. 26, 2019 (EP) 19188660.5

(51) Int. Cl. G01N 33/00

(52) U.S. Cl. CPC G01N 33/0075

(57) ABSTRACT

Provided is a method and apparatus for monitoring quality and determining contamination of a space, the method including measuring a plurality of quality parameter actual values in a space with a sensor system, wherein the actual value of at least one quality parameter is measured at a plurality of measuring locations, wherein the sensor system includes a plurality of sensors, comparing the quality parameter actual values with target values for the respective measuring locations, moving the at least one mobile sensor with a control system to a measuring location spaced apart from the previous measuring location of the at least one mobile sensor, wherein in the event of an incident at least one mobile sensor is moved with the control system deviating from the stored movement plan, wherein the data processing system determines the spatial spread, type of contamination and/or concentration of the respective contamination of the space.

### CROSS-REFERENCE TO RELATED APPLICATIONS

This application claims priority to PCT Application No. PCT/EP2020/070408, having a filing date of Jul. 20, 2020, based on European Application No. 19188660.5, having a filing date of Jul. 26, 2019, the entire contents both of which are hereby incorporated by reference.

### FIELD OF TECHNOLOGY

The following relates to a method for monitoring quality and detecting contamination of a space, and to a system for monitoring quality and detecting contamination of a space.

### BACKGROUND

A fluid in a space, for example the air in air spaces, for example above cities or industrial plants, can have different qualities depending on a variety of factors. For example, air quality impairment can be caused by local emissions of a variety of different substances or be wafted by wind. Such impairments may be perceptible to humans through odor or may be visually detectable. However, humans can usually only recognize such occurrences when the air quality has already been severely impaired. Some impairments cannot be recognized by humans at all, other impairments are very clearly perceptible but not dangerous. In addition, such impairments can be locally very limited, so that the impairment is only observable at a few locations.

Therefore, for the protection of the population, in some areas it is a need to monitor the quality of a space continuously and space-wide.

Systems for quality monitoring are generally known.

For example, US 2019/0113445 A1 describes an air pollution monitoring system and method, wherein a plurality of monitoring devices are arranged at different heights and levels, and infrared reflective arrays of the monitoring device are arranged at different heights to collect infrared spectral data of chemical constituents of the air mass diffusing in air spaces at different heights. The pollution factors contained in the air mass and the pollution level of each factor are determined using the IR spectrum for qualitative and quantitative analysis of the pollution factor, thereby enabling continuous monitoring in a specific area.

In order to monitor the quality with a high sensitivity and accuracy, a large number of sensors are usually used. However, these are sometimes very expensive and are therefore not installed to cover the entire space or at least not in a sufficient density. It can be time-consuming to install the various sensors at the different positions or to find suitable positions at all. Another disadvantage can be that, despite careful positioning, the sensors are blind to some locations and substances in the space or are distributed too coarsely. For a particularly precise determination of quality, sensors are also required that can only measure locally. Known methods and devices for monitoring the quality of spaces therefore still offer potential for improvement.

U.S. Pat. No. 7,383,129 B1 describes a system and method for visualizing a scene that is being monitored for substance contamination. The scene is scanned with a substance detector to detect a substance such as a harmful chemical or biological substance. The location of the substance is determined. Graphical element data is generated representing the detected substance in the scene. A displayed view of the scene is augmented with the graphical element data based on the position of the detected substance to display the detected substance in the view of the scene and indicate to the user the location and type of the detected substance in relation to the environment.

US 2009/018780 A1 describes a method and system for obtaining information about contaminants in ambient air. Multiple detection systems measure ambient air for contaminants in real time. Each of the multiple detection systems analyzes contaminants for hazardous substances. The multiple detection systems transmit information about the hazardous substances to a satellite monitoring system. The satellite monitoring system receives the transmitted information. The satellite monitoring system packages the information transmitted by each of the detection systems and analyzes the packaged information. The satellite monitoring system transmits the analyzed information to a console installed on a computing device at a command station. The console receives the transmitted information from the satellite monitoring system and communicates user interaction based on the transmitted information to the satellite monitoring system.

### SUMMARY

An aspect relates to an improved method and system for monitoring quality and detecting contamination of a space.

Embodiments of the invention propose a method for monitoring quality and detecting contamination of a space, wherein the space is a volume determinable by spatial coordinates, which comprises a fluid or which is substantially filled with a fluid.

The method comprises the steps,

a) measuring a plurality of quality parameter actual values in a space with a sensor system, wherein the space is a volume determinable by spatial coordinates, which comprises a fluid or which is substantially filled with a fluid, wherein the actual value of at least one quality parameter is measured at a plurality of measuring locations, wherein the sensor system comprises a plurality of sensors, wherein the sensors are mobile sensors, or at least one sensor is a stationary sensor and at least one sensor is a mobile sensor, wherein each sensor measures the at least one quality parameter actual value at a position of the measuring location defined by spatial coordinates,

b) transmitting the quality parameter actual values measured at a plurality of measuring locations to a data processing system,

c) comparing the quality parameter actual values measured at the respective measuring locations with target values stored in the data processing system for the respective measuring locations,

d) moving the at least one mobile sensor with a control system to a measuring location spaced apart from the previous measuring location of the at least one mobile sensor,
wherein the at least one mobile sensor moves to different measuring locations according to a movement plan stored in the data processing system or according to a newly calculated movement plan, wherein in the event of an incident in which at least one actual value deviates from the target value defined for the respective measuring location, at least one mobile sensor is moved with the control system deviating from the stored movement plan and, in the space around the position of the measuring location with the detected actual value deviation, measures additional quality parameter actual values at additional measuring locations and transmits them to the data processing system, wherein the data processing system determines the spatial spread, type of contamination and/or concentration of the respective contamination of the space.

For the purposes of embodiments of the present invention, a space is understood to be a volume that can be determined by spatial coordinates. The space may be partially or completely enclosed, for example by a floor, by walls and/or by a ceiling, but may also have no boundary. Under the term space it is to be understood that the space comprises at least one fluid or that the space is substantially filled with a fluid.

According to one embodiment, the space may comprise at least one fluid. According to a further embodiment, the space may be at least substantially filled with a fluid.

Substantially for purposes of embodiments of the invention means that the fluid occupies most of the volume of the space, or that more than 50 volume % of the space comprises a fluid.

A fluid in the sense of embodiments of the present invention is a gas, a liquid or a mixture comprising a gas and a liquid. In an embodiment, a fluid may comprise water, water vapor, and/or air. According to embodiments of the invention, for example, a fluid may comprise substantially water or air. In an embodiment, it may be provided that the space comprises an air space and/or the space comprises a water space, and the quality parameter is an air quality parameter and/or water quality parameter. In particular, it can be provided that the fluid of the space is in mass exchange with the fluid outside the space.

For the purposes of embodiments of the present invention, a number means a number of at least one. For the purposes of embodiments of the present invention, a plurality is to be understood as a number of at least two.

For the purposes of embodiments of the present invention, a quality parameter means a parameter that directly or indirectly indicates the quality of the medium in the space. In embodiments, it can be provided that the quality parameter is an air quality parameter and the space is an air space. Alternatively or additionally, it may be provided that the quality parameter is a water quality parameter, and the space is a body of water. In an embodiment, it may be provided that the quality parameter is selected from the group consisting of the concentration of a substance and/or a group of substances, the absorption and/or transmission of electromagnetic radiation by the fluid, and/or the strength of electromagnetic radiation per se. For example, concentrations of certain gases, liquids, or solids in the air may be understood as a quality parameter. In this context, measuring the quality parameter also can mean that the concentration is not measured directly, but another measurable parameter, for example an absorption, resistivity, capacitance or the like, which is then converted into the concentration as raw data. In this context, the unconverted raw data are also to be understood as quality parameters. For the purposes of embodiments of the present invention, a quality parameter may also be understood to mean a parameter that only indirectly influences the quality of the space. For example, concentrations of substances in a concentration range in which they do not directly influence the quality of the space can also be a quality parameter. Thus, this can also be understood to mean the detection of substances that serve, for example, to prepare/perform a terrorist attack, such as explosives, chemical, biological or radioactive substances (warfare agents), but also drugs or psychotropic drugs in small quantities. Thus, for the purposes of embodiments of the present invention, a contamination is not only to be understood as a contamination in the sense of a concentration that is hazardous to health, but also as a determinable deviation from a normal state.

For the purposes of embodiments of the present invention, an actual value means the value actually measured or the measured value converted from the raw data. For the purposes of embodiments of the present invention, a target value is to be understood as an acceptable value given for the actual value. Accordingly, the target value may be determined by an upper and/or lower limit value. Applicable limit values can be, for example, ERPG, MAK, IDLH, TEEL, AGW, AEGL, BAT, explosion limit values, radiation limit values, lethal doses, or lethal concentrations.

For the purposes of embodiments of the present invention, a measuring location is the location whose fluid is being measured. Accordingly, the measuring location can also have a corresponding measuring volume.

For the purposes of embodiments of the present invention, a quality parameter actual value means the actual value of the quality parameter at the measuring location where the quality parameter was measured.

Accordingly, a plurality of quality parameter actual values in the sense of embodiments of the present invention means at least two quality parameter actual values. At least two quality parameter actual values may differ sufficiently in the sense of embodiments of the present invention by the measured quality parameter itself and/or by the measuring location. For example, a plurality of quality parameter actual values may be achieved by measuring a quality parameter at two different measuring locations. Alternatively or additionally, a plurality of quality parameter actual values can be achieved by measuring different quality parameters at one measuring location. By measuring an actual value of at least one quality parameter at a plurality of measuring locations, a plurality of quality parameter actual values is accordingly already measured in the sense of embodiments of the present invention.

For the purposes of embodiments of the present invention, a mobile sensor is a sensor whose position in relation to space can be changed. It can be provided that the mobile sensor is not constantly in motion but can also hold its position at least temporarily. For the purposes of embodiments of the present invention, a stationary sensor is to be understood as a sensor whose position in relation to space cannot be changed in a regular manner. For example, this is to be understood as a sensor that is attached to a stationary object, such as a wall, a mast, a tower, a chimney or a street light. The attachment may be detachable, for example to allow maintenance or replacement of the stationary sensor.

Accordingly, in the method for monitoring quality and detection of contamination of a space in step a), a plurality of quality parameter actual values are measured in a space with a sensor system, the space being a volume determinable by spatial coordinates, which comprises a fluid or which is substantially filled with a fluid. The actual value of at least one quality parameter is measured at a plurality of measuring locations. Thus, an actual value for at least one quality parameter is measured at each measuring location, whereby overall an actual value is measured at different measuring locations and different quality parameters can also be measured at each measuring location. The sensor system comprises a plurality of sensors, wherein the sensors are mobile sensors, or at least one sensor is a stationary sensor and at least one sensor is a mobile sensor. Each sensor measures the at least one quality parameter actual value at a position of the measuring location determinable by spatial coordinates. In this context, a plurality of sensors means at least two sensors in the sense of embodiments of the present invention.

In an embodiment, a plurality of sensors may include, for example, ≥2 to ≤50, preferably ≥3 to ≤40, more preferably ≥4 to ≤35, even more preferably ≥5 to ≤30, further preferably ≥6 to ≤25, further preferably ≥7 to ≤20, mobile sensors. However, a plurality of sensors may also comprise, for example, ≥5 to ≤45, preferably ≥8 to ≤42, more preferably ≥9 to ≤38, even more preferably ≥10 to ≤36, more preferably ≥12 to ≤32, further preferably ≥15 to ≤18, mobile sensors.

In an embodiment, a plurality of sensors may include, for example, ≥1 to ≤50, preferably ≥2 to ≤40, more preferably ≥4 to ≤35, even more preferably ≥5 to ≤30, further preferably ≥6 to ≤25, further preferably ≥7 to ≤20, stationary sensors. However, a plurality of sensors may also comprise, for example, ≥3 to ≤45, preferably ≥8 to ≤42, more preferably ≥9 to ≤38, even more preferably ≥10 to ≤36, more preferably ≥12 to ≤32, further preferably ≥15 to ≤18, stationary sensors.

In an embodiment, a plurality of sensors can include, for example:
≥2 to ≤50, preferably ≥3 to ≤40, more preferably ≥4 to ≤35, still more preferably ≥5 to ≤30, more preferably ≥6 to ≤25, still more preferably ≥7 to ≤20, or ≥5 to ≤45, preferably ≥8 to ≤42, more preferably ≥9 to ≤38, even more preferably ≥10 to ≤36, more preferably ≥12 to ≤32, even more preferably ≥15 to ≤18, mobile sensors; and
≥1 to ≤20, preferably ≥2 to ≤18, more preferably ≥4 to ≤16, even more preferably ≥5 to ≤14, more preferably ≥6 to ≤12, further preferably ≥7 to ≤10 stationary sensors.

According to another embodiment, the ratio of stationary sensors to mobile sensors may be 1:3 to 1:100, preferably 1:4 to 1:60, more preferably 1:5 to 1:50, still more preferably 1:10 to 1:30, more preferably 1:15 to 1:20, further preferably 0:200 to 0:20000. For example, a swarm of 100 mobile sensors can be sent out to monitor a large area of 30×100 km, preferably 2×4 km.

In method step b), the quality parameter actual values measured at a plurality of measuring locations are transmitted to a data processing system. In this context, transmission is to be understood as systematic sending and receiving. It is understood that the quality parameter actual values are transmitted from the sensor system in such a way that they can be received, assigned and interpreted by the data processing system. The data processing system can consist of several data processing systems, which, for example, are coupled to the mobile and stationary sensor carrier systems, such as vehicle, aircraft, watercraft, preferably autonomously, or communicate with each other in a data network by systematic transmission and reception, and in total form a data processing system. In particular, the data processing system can assign the quality parameter actual values to the position of the measuring location determined by spatial coordinates. It can be provided that the sensor transmits the position of the measuring point directly to the data processing system. Alternatively, it can be provided that the sensor transmits its identity to the data processing system and the control system transmits the associated position to the data processing system. In the case of stationary sensors, it can be provided that the sensor only transmits its identity and the position of the measuring location is stored in the data processing system.

In method step c), the quality parameter actual values measured at the respective measuring points are compared with the target values stored in the data processing system for the respective measuring points. This determines whether the quality parameter actual values deviate from the target values.

In method step d), the at least one mobile sensor is then moved by a control system to a measuring location space apart from the previous measuring location of the at least one mobile sensor. Thus, after each method step d), the at least one sensor can measure at least one quality parameter actual value at a different measuring location.

It is provided that the at least one mobile sensor moves to various measuring locations according to a movement plan stored in the data processing system. The movement plan thus determines the movement of the at least one mobile sensor in each method step d). A movement plan adapted to the space to be monitored and to the number of mobile and stationary sensors can be stored.

It is provided that in case of an incident, in which at least one actual value deviates from the target value defined for the respective measuring location, at least one mobile sensor is moved with the control system deviating from the stored movement plan and is moved in the space around the position of the measuring location with the detected actual value deviation or preferably around a concentration cloud according to a dispersion prediction of the data processing system, measures additional quality parameter actual values at additional measuring locations and transmits them to the data processing system, whereby the data processing system determines the spatial dispersion, type of contamination and/or concentration of the respective contamination of the space and preferably predicts the dispersion again according to weather data. Accordingly, an incident is a case in which a deviation of a quality parameter actual value from the target value was determined in method step c). Thus, a contamination can be precisely determined.

An incident is a case in which at least one actual value deviates from the target value defined for the respective measuring point. The malfunction can be classified according to a hazard. For example, an incident can be a dangerous or a harmless incident. Depending on the hazard level of the incident, it may be provided that only information about the deviation is provided by the data processing system and that a movement deviating from the movement plan is not performed by a mobile sensor. For example, it can be provided that sand dust from the Sahara in Central Europe, spreading of fertilizer (manure, slurry) or grass cuttings/hay smell does not lead to a movement in the event of an incident.

The method described above advantageously enables a space to be monitored with a comparatively small number of sensors. The method described above advantageously enables the system itself to investigate in more detail if a quality parameter is exceeded, without having to rely on a particularly large number of sensors.

In an embodiment, it can be provided that the plurality of sensors comprises a number of stationary sensors and a number of mobile sensors, wherein at least one sensor is a stationary sensor and at least one sensor is a mobile sensor. In an embodiment, by an interaction of mobile and stationary sensors, it can be achieved that the method is particularly flexible and at the same time cost-efficient.

In an embodiment, it may be provided that the plurality of sensors comprises at least three sensors. In an embodiment, it may be provided that the plurality of sensors comprises a number of sensors adapted to the requirements of monitoring quality and detecting contamination of the space. For example, it may be provided that the plurality of sensors comprises at least four, five, six, seven, eight, nine, or ten sensors. For example, if the space to be monitored is particularly large or if the quality is to be monitored particularly densely, it may be provided that the plurality of sensors comprises at least twenty, thirty, forty, fifty, or one hundred sensors.

In an embodiment, it can be provided that at least two sensors are mobile sensors. In an embodiment, it may be provided that at least three, four, five, six, seven, eight, nine, or ten sensors are mobile sensors. In one embodiment, it may be provided that at least twenty, thirty, forty, fifty, or one hundred sensors are mobile sensors.

In an embodiment, it can be provided that at least one sensor is a stationary sensor. In a further embodiment, it may be provided that at least two, three, four, five, six, seven, eight, nine, or ten sensors are stationary sensors. In one embodiment, it may be provided that at least twenty, thirty, forty, fifty, or one hundred sensors are mobile sensors.

In one embodiment, the number of mobile sensors can be smaller than the number of stationary sensors. In an embodiment, this makes it possible to achieve a particularly simple and constant rough coverage of the space to be monitored. Furthermore, it can be achieved in this way that existing methods can be modified to the method according to embodiments of the invention with little effort. In an alternative embodiment, the number of mobile sensors can be greater than or equal to the number of stationary sensors. In an embodiment, this makes it possible to achieve particularly flexible monitoring with a particularly small number of sensors. This means, for example, that the first implementation of the method can be carried out particularly cost-effectively and quickly, since only a few or no stationary sensors need to be set up.

In one embodiment, it may be provided that the measuring location at which a mobile sensor measures a quality parameter actual value has a measurement volume that extends along a movement path of the mobile sensor. By this it is meant that the mobile sensor performs a measurement during a movement. For example, the mobile sensor may determine a measurement value for a certain time during the movement. In an embodiment, this may mean that the mobile sensor does not have to stop during the measurement. As a result, faster measurements are possible. In an embodiment, this also results in an averaged value for a larger measurement range, so that fewer measurements may be necessary to obtain good coverage of the space.

In an embodiment, it can be provided that the at least one quality parameter is the concentration of substances, in particular pollutants and/or hazardous substances, in the fluid, in particular in the air. The substances can be gases, liquids or solids that are dissolved and/or dispersed in the fluid, in particular in the air. The quality parameter may thus also concern aerosols or smoke. The quality parameter may concern the concentration of a single substance or single particles, or it may concern the concentration of a class of substances. The quality parameter can also describe a quality parameter of the fluid, in particular of the air, independently of specific substances. For example, the light absorption of the air can be a quality parameter without being specifically directed to the concentration of a substance. The quality parameter can also be the measured value of the sensor.

It may be provided that a sensor is a spectroscopic sensor. By this it is meant that the sensor measures the absorption, emission, scattering or diffraction of electromagnetic radiation, for example, radioactive radiation, X-ray radiation, ultraviolet radiation, visible light, infrared radiation and/or microwave radiation. For example, the sensor may be a Raman, infrared, or UV/Vis spectrometer. In embodiments, such sensors do not need to come into direct contact with the fluid, or air, or the substance or particle to be measured.

Furthermore, it can be provided that a sensor is a chemical sensor. This means that the sensor comes at least partially into contact with the substance to be measured and is influenced by the chemical and/or physical properties of the substance. For example, the resistance and/or the capacitance of a measuring layer of such a sensor may be influenced by the substance to be measured. In an embodiment, such sensors may be particularly selective for certain substances. In embodiments, a sensor may be a metal oxide semiconductor gas sensor, a solid state ion conductor and/or a photoionization detector. Alternatively or additionally, it may be provided that the sensor comprises a flame ionization detector, a nitrogen-phosphorus detector, a thermal conductivity detector, and/or an atomic emission detector.

It can be provided that a sensor additionally comprises a separation device. A separation device can be used to separate the measured fluid, in particular the measured air, into components before measuring an actual quality parameter value. For example, the air can be separated via chromatography, in particular gas chromatography. The air can alternatively or additionally be separated via an ion mobility spectrometer or alternatively or additionally via a mass spectrometer, in particular via a magnetic sector field or quadrupole mass spectrometer. Thus, it can be achieved that some substances can be more easily distinguished from each other by the sensors.

It can be provided that the sensor is adapted to perform individual analysis, i.e., to determine quality parameters in the sense of individual substances, and/or that the sensor is adapted to determine quality parameters in the sense of a sum parameter, such as TOC, the total content of organic material.

When the quality parameter concerns organic liquids that can be vaporized and organic gases, it may be desired that the sensor is a gas chromatograph coupled with at least one selected from the group of mass spectrometer (MS and/or MS/MS), flame ionization detector, nitrogen-phosphorus detector, thermal conductivity detector, electron capture detector, and atomic emission detector. When the quality parameter concerns liquids, it may be desired that the sensor be an HPLC-MS/MS with DAD and/or UV-Vis detector. For organic substances, inorganic compounds and/or liquids, it may be provided that the sensor is an NMR spectrometer, an infrared spectrometer and/or a Raman spectrometer. For organic and inorganic solutions and gases, it may be provided that the sensor is a UV-Vis spectrometer. If the quality parameter concerns aromatics, olefins, bromides and iodides, sulfides and mercaptans, organic amines, ketones, ethers, esters and acrylates, aldehydes, alcohols, alkanes, hydrogen sulfide, ammonia, and/or phosphanes, it may be desired that the sensor comprises a photoionization detector. If the quality parameter concerns organic and inorganic gases, explosives, drugs, and/or chemical warfare agents, it may be provided that the sensor is an ion mobility spectrometer. For inorganic and organic gases, it may be provided that the sensor comprises an electrochemical cell, test tubes, thermal sensors, conductivity sensors, electrochemical diffusion sensors, and/or a metal oxide sensor. In an embodiment, for measuring alpha, beta gamma, and neutron radiation, the sensor may comprise a radiation meter. Further, for wind speed measurement and position determination, it may be provided that the sensor comprises an ultrasonic and/or infrasonic measurement device. For determination of aerosols, dust, ozone, nitrogen oxides, methane, sulfur dioxide, wind speed and/or for object detection/environment detection, it may be provided that the sensor comprises a LIDAR. For object detection and/or environment detection, it may be provided that the sensor comprises a RADAR. If the quality parameter relates to dust, biomolecules and/or pollen, it may be provided that the sensor comprises a particle counting tube. For hazardous substances and halogenated compounds, for example chlorinated hydrocarbons, it may be desired that the sensor comprises a thermal catalytic surface ionization (TKOI) sensor. For oxygen content, the sensor may be an explosimeter. For humidity, the sensor may be a humidity sensor and/or capacitive sensor. For wind speed, the sensor may be a wind sensor and/or anemometer. For particles in liquids or gases, the sensor may include a turbidimeter and/or an optical sensor. For determining the refractive index of liquids or gases, the sensor may comprise a refractometer. Further, to determine temperature, the sensor may comprise a thermometer. For determining atmospheric pressure, the sensor may comprise a pressure sensor. Further, to determine pH, the sensor may comprise a pH meter. To determine salinity, the sensor may comprise a conductivity meter. If the quality parameter relates to biological aerosol particles and/or spores, the sensor may comprise a fluorescence meter. If the quality parameter relates to organic gases, the sensor may comprise a VOC sensor. If the quality parameter concerns inorganic gases, for example Li, Na, K, Ca, etc., the sensor may comprise a flame photometer. If the quality parameter relates to carbon dioxide and/or other IR active gases, it may be provided that the sensor comprises a solid state gas sensor (NDIR).

It can also be provided that a sensor is an optical image sensor. This means that an image of the space can also be recorded. The image sensor can also continuously record images of the space and in particular of the measuring location.

It may be provided to use one sensor to measure different quality parameters at one measuring location. This means that the sensor can be adapted to measure different quality parameters. For this purpose, in embodiments it can be provided that the sensor comprises different measuring systems, each measuring system being adapted to measure at least one quality parameter. For example, the sensor may be a spectroscopic sensor and/or a chemical sensor. By this is to be understood that the sensor comprises a measuring system that is adapted to perform spectroscopic measurements and a measuring system that is adapted to perform chemical measurements.

According to an embodiment, it may be provided that the sensor system comprises additional mobile sensors that do not follow a movement plan and are not controlled by the control system. For example, the sensor system may include sensors worn by people, such as personal dosimeters or sensors for workplace monitoring, such as gas detectors, or sensors attached to vehicles that are not controlled by the control system. These sensors may be equipped with a positioning device and may indicate the associated measuring location in addition to measurement values.

According to an embodiment, it may be provided that the measurement of a quality parameter actual value comprises a sample collecting. Sample collecting (or sampling) is understood to mean the collection of a sample, in particular the collection and taking away of a sample. In this context, it can be provided that the sample collecting is carried out with a passive sampler, which can lower the detection limit of the system due to the longer exposure time. It may be provided that the passive collector is stationary and is collected, measured and evaluated by a mobile sensor. The stationary system can then be equipped with a new or the “reset” passive collector by the mobile sensors. According to an embodiment of the invention, it may be provided that the mobile sensor takes a sample and transfers it to another sensor for analysis. For example, it may be provided that a flying mobile sensor takes a sample and transfers it to a sensor traveling on the ground or a stationary sensor. The ground traveling sensor or stationary sensor can then analyze the sample and transmit the actual quality parameter value to the data processing system. In an embodiment, this allows complicated measurement methods to be used. In particular, the mobile sensors can thus be kept small. It can also be achieved that the number of sensors, in particular particularly expensive sensors, can be reduced, since several mobile sensors can deliver their fluid samples to a single sensor.

In an embodiment, it may be provided that sample collecting is performed with a collection system selected from the group consisting of solid phase microextraction fibers, “gas mice”, activated carbon tubes, Tenax® tubes, reservoir vessels for gases and/or liquids.

In an embodiment, sample collecting can be initiated in the event of the detection of unknown substances or an increase in certain actual quality parameter values that cannot be assigned to any substance without additional measurement. Alternatively or additionally, permanent sample collecting with passive samplers can be provided, which are collected/measured at regular intervals. It can be provided in an embodiment to measure the samples by the mobile sensor units. Alternatively or additionally, it can be provided to transport the samples with the mobile sensors to the place of analysis, e.g., outside hazardous areas. The transport path through the air by the mobile sensors can thereby significantly reduce the time until the results are obtained. In an embodiment, provision may be made for transport to a central and/or specialized sensor. In an embodiment, passive collectors can be installed instead of sensors at locations with a low probability of occurrence.

According to an embodiment, it may be provided that the sample collection also comprises a collection of reserve samples. In an embodiment, it can be provided that the mobile sensors take the samples and transfer them to a storage system. In this way, proof of the quality of the space, in particular of an air space, can be provided if necessary.

The movement plan can provide a sequence of measuring locations for each mobile sensor. The measuring locations can advantageously be selected so that they cover the space as homogeneously as possible. The measuring locations can be selected closer to each other in certain areas of the space, for example sources of danger. The movement plan can also be a schedule, so that the mobile sensor is moved to the various measuring locations at a specific time interval. It may be provided that the movement schedule also provides for a schedule according to certain times of the day. For example, certain areas of the space may provide for lower spaced measuring locations at certain times of the day than at other times of the day. In addition, the movement schedule may take into account the number of mobile sensors and/or the number of stationary sensors. The movement plan may provide the same measuring locations for each mobile sensor, with the mobile sensors being moved to the designated measuring locations in a staggered manner. Alternatively, the movement plan may provide for different measuring locations for different mobile sensors. For example, the movement plan may provide a separate movement plan for each mobile sensor.

The movement plan can thus be particularly well adapted to the given conditions of the space and the sensor system. Thus, efficient monitoring of the space can be achieved particularly easily. The monitoring can also be adapted particularly easily to changes in the sensor system, the space and/or the environment of the space.

In the event of an incident, at least one mobile sensor with the control system deviates from the stored movement plan. In particular, it can be provided that the mobile sensor which is in the event of an incident the least distant from the measuring locating of the actual value deviating from the target value is moved around the position of the measuring location with the detected actual value deviation. In an embodiment, it can be provided that several or all mobile sensors are moved around the position of the measuring location with the detected actual value deviation. Depending on the type of quality parameter and the size of the deviation, an urgency can be assigned to the incident case. Depending on the urgency, a number of mobile sensors used can then be selected, whereby all mobile sensors can be used in the case of an incident with the highest urgency and at least one mobile sensor is used in the case of an incident with the lowest urgency. The sensor system can also have additional mobile sensors that are not currently following the movement plan, for example sensors that are currently on call. In the event of an incident, these additional mobile sensors can also be deployed. In addition, in the event of an incident, it may be provided that a stationary sensor increases its measurement frequency. For example, it can be provided that a stationary sensor that performs measurements in a certain time interval performs measurements in a smaller time interval in the event of an incident. It can also be provided that a stationary sensor is in a stand-by mode and only performs measurements in the event of an incident.

In this way, it can be achieved that the contamination of the space can be precisely determined in the event of an incident. By using the existing mobile sensors, on the one hand, it is possible to react very quickly to contamination of the space and, on the other hand, resources can be saved because additional emergency systems for determining the contamination can be dispensed with.

The movement around the position of the measuring point with the detected deviation can be carried out in a step according to a localization plan. The mobile sensors used can be systematically moved around the measuring location with the detected deviation. In another step, the movement around the position of the measuring location can be adjusted according to the quality parameter actual values measured in the method. For example, the sensors can be moved along increasing quality parameter actual values or along constant or decreasing quality parameter actual values.

Thus, it can be achieved that the exact spread of the contamination can be determined. In addition, the course of the contamination over time can be determined and, if necessary, the source of the contamination can be identified.

According to an embodiment of the method, it can be provided that in step a) additional fluid parameters, in particular air parameters, in particular wind direction, wind force, air temperature, air humidity and/or air pressure are measured and transmitted to the data processing system in step b). In an embodiment, it can be provided that the movement plan is adapted to the further fluid parameters, in particular air parameters. For example, it can be provided that the measuring locations of the movement plan are shifted according to a measured wind direction and wind strength.

This means that the measuring locations can be selected particularly efficiently. Thus, a particularly efficient air monitoring can be achieved.

According to an embodiment, it may be provided that the at least one quality parameter actual value is the concentration of a substance in the fluid, in particular in the air, in particular the concentration of a hazardous substance and/or pollutant.

For example, the quality parameter actual value can be the concentration in the air of ammonia, non-methane volatile organic compounds (NMVOC), carbon monoxide, sulfur dioxide, dust (with special consideration of the PM10 and PM2.5 fractions), nitrogen oxides, persistent organic pollutants (POPs), and heavy metals. The quality parameter actual value can also be the concentration of a substance that occurs more frequently in the vicinity of the measured value. For example, around corresponding industrial facilities, the quality parameter actual value can be the concentration of substances used in the industrial facility, especially exhaust gases, emissions and/or hazardous substances possibly emitted by the industrial facility.

According to an embodiment, it may be provided that further environmental data is made available to the data processing system, in particular information about possible emission sources.

This means that the movement plan can be adjusted accordingly. In addition, the environmental data can be used in the event of an incident to search more specifically for a cause for the target value deviation.

According to an embodiment, the further environment data can be provided to the data processing system automatically, semi-automatically and/or manually. Automatically provided further environmental data can, for example, be continuously made available to the data processing system from external sources. For example, it may be provided that information about the weather or about the surrounding traffic situation is continuously made available to the data processing system. Semi-automatically provided further environmental data may be information that is provided to the data processing system only in certain cases, either automatically or manually triggered. For example, operational disturbances can be automatically or manually transmitted to the data processing system. In an embodiment, manually provided further environment data may be further environment data that is not previously classified. For example, observations of the population may be provided to the data processing system. For example, odors or smoke may be noticed by residents and communicated to the data processing system by telephone calls to a central office. For this purpose, it is provided that the data processing system comprises an input interface via which information about further environmental data can also be communicated manually to the data processing system.

The environment data provided can also be dynamic environment data, in particular in real time. For example, data on road, ship or air traffic can be provided. In this context, dynamic environmental data means, in particular, information about possible emission sources that can be changed. For example, it may mean information about natural or technical events that have an impact on quality. For example, information about traffic congestion from nearby roads, road marking work, construction sites, volcanic eruptions and/or sandstorms can be dynamic environmental data. Alternatively or additionally, data about production schedules of corresponding industrial facilities can be provided, for example. This may include information about substances that may be emitted. Thus, the movement plan can be modified according to the environmental data. For example, mobile sensors, quality parameters to be monitored, and measuring locations can be selected according to the expected type and position of emissions.

In an embodiment, it can be provided that the movement plan comprises stationary objects with their spatial coordinates to which a minimum distance and/or maximum distance for the approach of the mobile sensor is stored. This means that the movement plan can provide movement corridors or areas into which the mobile sensor should or must not be moved under any circumstances.

In this way, it can be achieved in particular that the movement of the mobile sensor can be simplified by the control system. In particular, stationary objects can be taken into account so that collisions with these objects can be prevented without much effort. In addition, it can be prevented that mobile sensors that are not sufficiently protected are moved, for example, into explosive areas. Thus, the safety of the method can be increased.

According to an embodiment, it can be provided that measuring locations are defined in the movement plan, which at least one mobile sensor in a predetermined sequence or statistically heads to; and/or at least one mobile sensor/s randomly selects the measuring locations within the space to be monitored.

By using a predefined sequence, it can be achieved that a minimum monitoring can be easily guaranteed. Randomly selected measuring locations can minimize statistical errors. In particular, it can be provided that, in the case of a plurality of mobile sensors, some mobile sensors control defined measuring locations in a predefined sequence and other mobile sensors randomly select measuring locations within the space to be monitored.

In the case of a plurality of mobile sensors, the movement plan may provide for the greatest possible spatial distribution of the mobile sensors. This means that the sensors are distributed particularly evenly over the space to be monitored.

According to an embodiment, it can be provided that the movement plan is dynamically adapted. In this way, it is possible to ensure that the movement plan permits optimum monitoring of the quality parameters in any given situation. In an embodiment, the dynamic adaptation of the movement plan can be an adaptation to the environmental data. In this context, it may be provided that the movement plan provides for a greater measurement frequency or a greater measurement density at some locations in space. For example, it may be provided that the movement plan provides for a greater measurement frequency at locations with a greater probability of an incident occurring. In an embodiment, it can also be provided that the movement plan provides for a greater measurement frequency at locations where a potential incident case poses a greater danger. Furthermore, it may be provided that adapting the movement plan to the environmental data comprises adapting to the wind direction and/or the weather. The adapting may also be an adapting to the time of day, the day of the week, the month and/or the season.

According to an embodiment, it can be provided that the adaptation of the movement plan is based on historical measurement data of the method and environmental data. In an embodiment, an artificial intelligence can be used that is adapted to adapt the movement plan from historical measurement data and environmental data in order to obtain a particularly efficient monitoring of the space. For example, it can be provided that areas are identified that have a particularly high risk of an incident or a particularly high incident frequency, and the movement plan is automatically modified to obtain a particularly dense monitoring of these areas in terms of time and location. In this way, it can be achieved that the method adapts better and better to the conditions of the space over the period of use and can also react to changes. For example, the movement plan can thus be automatically adapted to new sources of danger, such as new or aged production equipment.

According to an embodiment, it can be provided that at least one mobile sensor determines the location of the highest concentration of the substance above the target value in the fluid, in particular in the air, in the event of an incident.

In particular, this makes it possible to determine whether the emission continues to persist. In addition, it can be tracked at which location the greatest danger is present and in which direction this location may be moving. This means that appropriate safety measures can be taken in a particularly targeted manner.

According to an embodiment, it may be provided that the quality parameter actual values transmitted by the stationary sensor(s) and/or mobile sensor(s) to the data processing system are processed by the data processing system to create a false color map taking into account the target values.

This can ensure that the information obtained through aerial surveillance can be well understood, reported, and further processed.

According to an embodiment, it may be provided that the data processing system extrapolates the quality parameter actual value in time and/or space on the basis of at least one quality parameter actual value and further air data.

This can ensure that the source of an emission can be easily found. In addition, it can be achieved that protective measures can be initiated particularly proactively and quickly. In an embodiment, the system itself initiates the protective measures.

According to an embodiment, a marker substance can be applied in the event of a malfunction. A marker substance is understood to be an essentially harmless substance that can be easily measured qualitatively and, in an embodiment, also quantitatively by sensors. The marker substance can then be measured like an actual quality parameter value in the event of a malfunction. In this way, it can be achieved that sensors that are not equipped to measure the exceeded quality parameter actual value can also track a spread via the marker substance. The marker substance can, for example, also be perceptible, in particular visible, to humans.

According to an embodiment, it can be provided that the data processing system issues automatic warnings when the actual value exceeds the target value and depending on the spread and type of exceedance of the actual value from the target value, whereby the data processing system gives an all-clear signal when the actual quality parameter value(s) correspond to the target value(s) stored in the data processing system for the respective space examined by stationary sensor(s) and/or mobile sensor(s).

For example, the data processing system can forward warnings to warning apps. In addition, the data processing system can inform emergency services such as the fire department or civil protection, or even the responsible private security services.

This can ensure that help can be provided more quickly if necessary.

According to an embodiment, it can be provided that, if the actual value exceeds the target value, the data processing system proposes and/or carries out hazard prevention measures in accordance with an action plan stored in the data processing system in this respect, in particular a shutdown of possible emission sources and/or a blocking off of hazard areas.

This is particularly intended for sources that are particularly hazardous and easy to control. For example, it could be stored in the data processing system that a specific production plant can emit specific pollutants in the event of a malfunction. In the stored action plan, it can be stored that the production plant is shut down if the target value of the specific pollutant is exceeded.

Further measures can be the closing of ventilation openings, the switching on of exhaust air systems, the triggering of warning systems, the actuation, in particular the closing, of pressure relief devices, expansion devices, emergency valves and emergency dampers. This can ensure that particularly hazardous emissions are stopped as quickly as possible.

According to an embodiment, it can be provided that the mobile sensor, depending on the respective measuring location and based on the measured quality parameter actual value, sends this data to the data processing system for the creation of a target value profile for different measuring locations of the space.

This means that a target value can also be below a usual limit value. Thus, a deviation can be detected more quickly by the method without excessively increasing the number of false alarms. In particular, by creating a target value profile, it can be achieved that even very small deviations from the target value profile can be measured. Such deviations can then be followed up separately, for example. In this way, even small deviations can provide indications of material fatigue or impending emissions. Preventive measures can thus be initiated in the event of deviations from the nominal target value profile.

Embodiments of the invention further propose a system for monitoring quality and detecting contamination of a space, wherein the space is a volume determinable by spatial coordinates, which comprises a fluid or which is substantially filled with a fluid.

The system comprises a sensor system, a data processing system and a control system, wherein the sensor system comprises a plurality of sensors, wherein the sensors are mobile sensors, or at least one sensor is a stationary sensor and at least one sensor is a mobile sensor, wherein the sensor system is adapted to measure a plurality of quality parameter actual values in a space, wherein the space is a volume determinable by spatial coordinates, which comprises a fluid or which is substantially filled with a fluid, wherein the actual value of at least one quality parameter is measured at a plurality of measuring locations, wherein each sensor measures the at least one quality parameter actual value at a position of the measuring location defined by spatial coordinates, wherein the sensor system is adapted to transmit the quality parameter actual values measured at a plurality of measurement locations to the data processing system, wherein the data processing system is adapted to compare the quality parameter actual values measured at a plurality of measuring locations with target values provided for the respective measuring location, wherein the control system is adapted to move the at least one mobile sensor to a measuring location space apart from the previous measuring location of the at least one mobile sensor, wherein the at least one mobile sensor moves to different measuring locations according to a movement plan stored in the data processing system, characterized in that, in the event of an incident, in which at least one actual value deviates from the target value defined for the respective measuring location, at least one mobile sensor is moved with the control system deviating from the stored movement plan and, in the space around the position of the measuring location with the detected actual value deviation, measures additional quality parameter actual values at additional measuring locations and transmits them to the data processing system, the data processing system determining the spatial spread, type of contamination and/or concentration of the respective contamination of the space.

In an embodiment, the system described above can be used to monitor a space particularly efficiently and to detect contamination of the space particularly precisely and quickly. The system thus serves to carry out the method described above.

According to an embodiment, it can be provided that the data processing system comprises computers or data processing devices that can communicate with each other, whereby it can be provided that a computer or data processing device is arranged on the at least one mobile sensor. The data processing system can consist of several data processing systems, which are coupled, for example, to the mobile and stationary sensor carrier systems, such as vehicle, aircraft, watercraft, preferably autonomously, or which communicate with one another in a data network by systematic transmission and reception and, in total, form a more efficient data processing system.

According to an embodiment, it may be provided that the mobile sensor is a ground vehicle, rail vehicle, cable car vehicle, water vehicle and/or an aircraft, wherein the system in particular comprises at least two mobile sensors and one mobile sensor is a ground vehicle, rail vehicle, cable car vehicle or water vehicle and another mobile sensor is an aircraft, in particular a drone. By providing that a sensor is a particular vehicle, it is understood that a corresponding sensor is arranged on a corresponding vehicle. Thus, in the sense of embodiments of the present invention, a mobile sensor is also a vehicle having a fixedly arranged sensor.

Ground vehicles make it particularly easy to implement the system. Ground vehicles are known and particularly safe in themselves, as they usually simply stop in the event of malfunctions. However, for space-covering air monitoring, it is desirable that measuring points are also located well above the ground. It may therefore be provided that the ground vehicle comprises superstructures designed to reach measuring locations at different heights, for example telescopic arms.

Mobile sensors can also include vehicles. In an embodiment, these vehicles are equipped with a navigation system that, in the event of an incident, provides driving instructions to the driver to take new measurements at a safe distance or to move away from the hazardous area. In an embodiment, autonomous vehicles that can be steered into the hazardous area in the event of an incident and take measurements to provide an improved propagation forecast and more accurate information about the hazardous zones in the situation picture.

Aircraft can be used to reach measuring locations at different heights particularly well. Particularly preferred aerial vehicles are multicopters and/or airships, for example with attached sensors, optionally on a rope at different heights. Such aerial vehicles can advantageously be easily controlled and, in particular, stand in the air. In this way, it is advantageous that measurements can be taken at almost any measuring location and at any measuring height.

Water vehicles can be used to reach measuring locations at different depths in waters and seas particularly well and to supply them with energy over a long period of time. Water vehicles with attached sensors, for example on a rope, at different depths are particularly preferred. Advantageously, such watercraft can be easily steered and, in particular, stand in the body of water. In this way, it can be achieved that measurements can be taken at almost any measuring location and at any measuring depth, for example oxygen content, CO2 concentration, etc.

According to an embodiment, it may be provided that the ground vehicle, rail vehicle, cable car vehicle, water vehicle and/or the air vehicle is autonomously operable, in particular in autonomy levels 4 or 5. For example, the vehicles may be UAV's, UGV's, UWV's, UUV's, AUV's or USV's.

As a result, the ground vehicle, rail vehicle, cable car vehicle, water vehicle and/or the air vehicle can be particularly small. In addition, the vehicles can advantageously be controlled centrally. Automated control can also be provided. Thus, it can be provided that the sensors automatically follow the movement plan.

According to an embodiment, it may be provided that the aircraft can be transported by a ground vehicle, rail vehicle, cable car vehicle and/or watercraft. The ground vehicle or watercraft may also be a mobile sensor. Thus, the aircraft can be launched from the ground vehicle or watercraft. This allows areas that are difficult or impossible to reach or obstructed by vehicles to be explored and measurements to be taken. This can simplify the complexity of controlling the mobile sensors.

It may be provided that the mobile sensor have navigation sensors. In particular, it may be provided that the mobile sensor comprises at least one of GPS, radar system, LIDAR, laser, and optical sensors. Thus, the sensor may provide data required for navigation by itself. It may be provided that navigation data is provided to the sensor. The sensor may use the provided navigation data and navigation sensors for navigation.

It can be provided that the mobile sensor is explosion-proof. This means that the mobile sensor can also be used in potentially explosive atmospheres.

Embodiments of the invention further propose the use of the method for monitoring quality and detecting contamination of a space, wherein the space is a volume determinable by spatial coordinates, which comprises a fluid or which is substantially filled with a fluid. It may be provided to use the method for monitoring:
Chemical and industrial sites and plants/operations/facilities (e.g., refineries, warehouses, wastewater treatment plants, pipelines (above ground, buried, laid in bodies of water),
Transport infrastructure (e.g., roads, ports, airports, train stations, railroad tracks, . . . ),
Municipal areas, public institutions (authorities, kindergartens, schools, universities, research centers, hospitals . . . ),
Critical infrastructure, utilities (e.g., pipeline, canals, waterways),
Military facilities, radiation protection areas,
Cultural properties, nature reserves, protected landscape areas, world heritage sites, animal protection areas, for example, sensor systems can also be carried by animals, natural emission sources such as underground coal fires, oil spills, volcanoes, etc.,
Human and animal settlement areas, coastal areas, events (sports, culture, etc.),
Fattening farm sites, livestock farms, pasture sites,
Waters (oil spills, microplastics).

New Year's Eve activities, fire games, fire displays, volcanic activities, forest fires, desert storms, storms with erosion effects, snowstorms, pollen counts (allergy sufferers), dust pollution, spray from e.g., pesticides.

Although the present invention has been disclosed in the form of preferred embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

1. A method for monitoring quality and detecting contamination of a space, comprising:
a) measuring a plurality of quality parameter actual values in the space with a sensor system, wherein the space is a volume determinable by spatial coordinates, which comprises a fluid or which is substantially filled with the fluid, wherein the actual value of at least one quality parameter is measured at a plurality of measuring locations, wherein the sensor system comprises a plurality of sensors, wherein the sensors are mobile sensors, or at least one sensor is a stationary sensor and at least one sensor is a mobile sensor, wherein each sensor measures the at least one quality parameter actual value at a position defined by spatial coordinates,
b) transmitting the quality parameter actual values measured at the plurality of measuring locations to a data processing system,
c) comparing the quality parameter actual values measured at respective measuring locations with target values stored in the data processing system for the respective measuring locations,
d) moving the at least one mobile sensor with a control system to a further measuring location spaced apart from the respective measuring location of the at least one mobile sensor,
wherein the at least one mobile sensor moves to different measuring locations according to a movement plan stored in the data processing system,
wherein
in the event of an incident in which at least one actual value deviates from the target value defined for the respective measuring location, at least one mobile sensor is moved with the control system deviating from the stored movement plan and, in the space around the position of the measuring location with the detected actual value deviation, measures additional quality parameter actual values at additional measuring locations and transmits them to the data processing system, wherein the data processing system determines the spatial spread, type of contamination and/or concentration of the respective contamination of the space.

2. The method according to claim 1,
wherein in step a) additional further parameters, in particular wind direction, wind strength, air temperature, air humidity and/or air pressure are measured, and in step b) are transmitted to the data processing system.

3. The method according to claim 1, wherein the at least one quality parameter actual value is the concentration of a substance in the fluid, in particular the air, in particular the concentration of a hazardous substance and/or pollutant.

4. The method according to claim 1, wherein further environmental data is provided to the data processing system, in particular information about possible emission sources.

5. The method according to claim 1, wherein measuring a quality parameter actual value comprises sample collecting.

6. The method according to claim 1, wherein the movement plan comprises stationary objects with their spatial coordinates to which a minimum distance and/or maximum distance for the approach of the mobile sensor is stored.

7. The method according to claim 1, wherein
a) measuring locations are defined in the movement plan, which at least one mobile sensor in a predetermined sequence or statistically heads to; and/or
b) at least one mobile sensor randomly selects the measuring locations within the space to be monitored.

8. The method according to claim 1, wherein at least one mobile sensor determines the location of the highest concentration of the substance above the target value in the space in the event of an incident.

9. The method according to claim 1, wherein the quality parameter actual values transmitted from the stationary sensor(s) and/or mobile sensor(s) to the data processing system are processed by the data processing system to produce a false color map taking into account the target values.

10. The method according to claim 1, wherein the data processing system extrapolates the quality parameter actual value in time and/or space on the basis of at least one quality parameter actual value and further data.

11. The method according to claim 1, wherein the data processing system issues automatic warnings when the actual value exceeds the target value and depending on the spread and type of exceedance of the actual value from the target value, wherein the data processing system gives an all-clear signal when the quality parameter actual value(s) correspond to the target value(s) stored in the data processing system for the space examined by stationary sensor(s) and/or mobile sensor(s).

12. The method according to claim 1, wherein the data processing system, if the actual value exceeds the target value, proposes and/or carries out hazard prevention measures in accordance with an action plan stored in the data processing system.

13. The method according to claim 1, wherein the mobile sensor, depending on the respective measuring location and based on the measured quality parameter actual value, sends this data to the data processing system for creating a target value profile for different measuring locations of the space.

14. A system for monitoring quality and detecting contamination of a space, wherein the space is a volume determinable by spatial coordinates, which comprises a fluid or which is substantially filled with a fluid, comprising: a sensor system, a data processing system, and a control system,
wherein the sensor system comprises a plurality of sensors, wherein the sensors are mobile sensors, or at least one sensor is a stationary sensor and at least one sensor is a mobile sensor, wherein the sensor system is configured to measure a plurality of quality parameter actual values in the space, wherein the actual value of at least one quality parameter is measured at a plurality of measuring locations, wherein each sensor measures the at least one quality parameter actual value at a position defined by spatial coordinates,
wherein the sensor system is configured to transmit the quality parameter actual values measured at the plurality of measuring locations to the data processing system,
wherein the data processing system is configured to compare the quality parameter actual values measured at the plurality of measuring locations with target values provided for respective measuring location,
wherein the control system is configured to move the at least one mobile sensor to a further measuring location spaced apart from the respective measuring location of the at least one mobile sensor,
wherein the at least one mobile sensor moves to different measuring locations according to a movement plan stored in the data processing system,
wherein
in the event of an incident in which at least one actual value deviates from the target value defined for the respective measuring location, at least one mobile sensor is moved with the control system deviating from the stored movement plan and, in the space around the position of the measuring location with the detected actual value deviation, measures additional quality parameter actual values at additional measuring locations and transmits them to the data processing system, the data processing system determines the spatial spread, type of contamination and/or concentration of the respective contamination of the space.

15. The system according to claim 14, wherein the mobile sensor is a ground vehicle, rail vehicle, cable car vehicle, water vehicle and/or an air vehicle, wherein the system in particular comprises at least two mobile sensors and one mobile sensor is a ground vehicle, rail vehicle, cable car vehicle or a water vehicle and another mobile sensor is an air vehicle.

16. The system according to claim 15, wherein the ground vehicle, rail vehicle, cable car vehicle, water vehicle and/or the air vehicle is autonomously operable.

17. A use of the method according to claim 1 for quality monitoring and detection of contamination of the space.
